(19) United States
(12) Patent Application Publication
(10) Pub. No.: US 2025/0232448 A1
(43) Pub. Date: Jul. 17, 2025

(54) ULTRASONIC IMAGE PROCESSING APPARATUS, NON-TRANSITORY COMPUTER-READABLE STORAGE MEDIUM STORING ULTRASONIC IMAGE PROCESSING PROGRAM, AND ULTRASONIC IMAGE PROCESSING METHOD

(71) Applicant: CANON MEDICAL SYSTEMS CORPORATION, Otawara-shi (JP)

(72) Inventors: Chihiro SHIBATA, Nasushiobara (JP); Minori OHASHI, Otawara (JP); Akihiro KAKEE, Nasushiobara (JP); Kuramitsu NISHIHARA, Otawara (JP)

(73) Assignee: CANON MEDICAL SYSTEMS CORPORATION, Otawara-shi (JP)

(21) Appl. No.: 19/015,945

(22) Filed: Jan. 10, 2025

(30) Foreign Application Priority Data
Jan. 15, 2024 (JP) 2024-004020

(51) Int. Cl. G06T 7/00; A61B 8/00; A61B 8/06

(52) U.S. Cl. CPC G06T 7/0016; A61B 8/06; A61B 8/461; A61B 8/488; A61B 8/54; G06T 2200/24; G06T 2207/30104

(57) ABSTRACT

In one embodiment, an ultrasonic image processing apparatus includes processing circuitry. The processing circuitry is configured to: acquire a plurality of ultrasonic images in a prescribed period; and select a first ultrasonic image of a specific time phase from the plurality of ultrasonic images in the prescribed period based on a selection standard which is preset.

### CROSS-REFERENCE TO RELATED APPLICATION

This application claims the benefit of priority of Japanese Patent Application No. 2024-4020, filed on Jan. 15, 2024, the entire contents of which are incorporated herein by reference.

### FIELD

Disclosed embodiments in the present description and drawings relate to an ultrasonic image processing apparatus, a non-transitory computer-readable storage medium storing an ultrasonic image processing program, and an ultrasonic image processing method.

### BACKGROUND

In an examination by an ultrasonic diagnostic device, ultrasonic images may be preserved as moving images or still images and utilized for diagnosing or saved as records. Since a moving image is a series of frame images, in a case of preservation as a moving image, a plurality of ultrasonic images having a time width is stored. On the other hand, since a still image is an image of one frame, in the case of preservation as a still image, it is desirable that an ultrasonic image of a specific time phase suitable for an imaging region and an examination content is selected and preserved.

A user such as an examiner or an engineer performs, for example, a freeze operation while referring to ultrasonic images displayed on a display in real time during an examination, in order to preserve the ultrasonic image of the specific time phase as a still image. The freeze operation is an operation for stopping display of the ultrasonic images acquired in real time. However, there may be a time lag between a timing at which the user wants to perform the preservation as the still image and a timing of the freeze operation by the user, and the still image displayed by the freeze operation may not show the specific time phase but afterwards. In this case, the user traces back and confirms the plurality of still images from the still image displayed by the freeze operation using a track ball or the like, and selects and preserves the ultrasonic image of the specific time phase that the user wants to preserve as the still image.

If the user needs to perform such confirmation work when preserving the ultrasonic image of the specific time phase as the still image, it takes time for the confirmation work and examination efficiency declines. In addition, in the case where the user manually performs tracing back to the specific time phase, there may be user's subjectivity in selection of the still image, and the ultrasonic image to be preserved as the still image may be different depending on an experience level or the like of the user.

### BRIEF DESCRIPTION OF THE DRAWINGS

FIG. 1 is a block diagram showing a configuration example of an ultrasonic diagnostic device including an ultrasonic image processing apparatus according to an embodiment;

FIG. 2 is a block diagram showing a configuration example of processing circuitry of the ultrasonic image processing apparatus according to a first embodiment;

FIG. 3 is a flowchart showing an operation example of the ultrasonic image processing apparatus according to the first embodiment;

FIG. 4 is an explanatory diagram showing image selection condition examples in the first embodiment;

FIG. 5A to FIG. 5D are explanatory diagrams about selection of a first ultrasonic image in a carotid artery;

FIG. 6A to FIG. 6D are explanatory diagrams about the selection of the first ultrasonic image in a circulatory organ;

FIG. 7 is a flowchart showing an operation example of the ultrasonic image processing apparatus according to a modification of the first embodiment;

FIG. 8 is a block diagram showing a configuration example of the processing circuitry of the ultrasonic image processing apparatus according to a second embodiment;

FIG. 9 is a flowchart showing an operation example of the ultrasonic image processing apparatus according to the second embodiment; and

FIG. 10 is a flowchart showing an operation example of the ultrasonic image processing apparatus according to a third embodiment.

### DETAILED DESCRIPTION

Hereinafter, embodiments of an ultrasonic image processing apparatus, a non-transitory computer-readable storage medium storing an ultrasonic image processing program, and an ultrasonic image processing method will be described in detail with reference to the drawings. In the drawings, same elements are denoted by same signs and redundant description is omitted.

In one embodiment, an ultrasonic image processing apparatus includes processing circuitry. The processing circuitry is configured to: acquire a plurality of ultrasonic images in a prescribed period; and select a first ultrasonic image of a specific time phase from the plurality of ultrasonic images in the prescribed period based on a selection standard which is preset.

### (Ultrasonic Diagnostic Device)

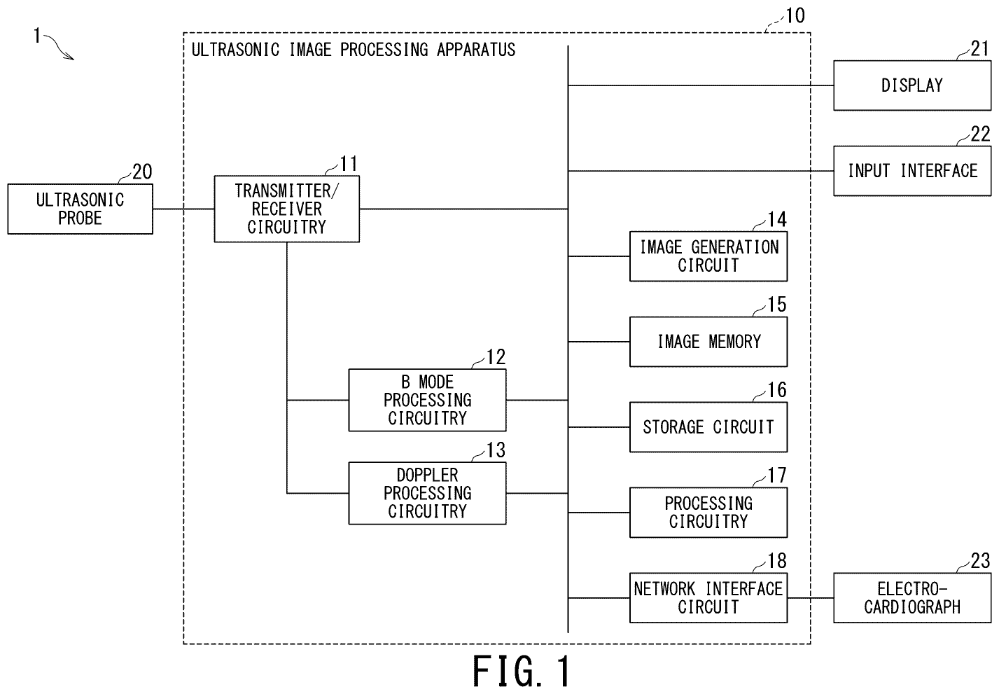

FIG. 1 is a block diagram showing a configuration example of an ultrasonic diagnostic device 1 including an ultrasonic image processing apparatus 10 according to the embodiment. The ultrasonic diagnostic device 1 includes the ultrasonic image processing apparatus 10, an ultrasonic probe 20, a display 21, an input interface 22, and an electrocardiogram a stationary device, the ultrasonic image processing apparatus 10 may include the display 21 and the input interface 22. In addition, the ultrasonic image processing apparatus 10 may form a tablet type or a smartphone type which has the display 21 and the input interface 22.

The ultrasonic image processing apparatus 10 includes transmitter/receiver circuitry 11, B mode processing circuitry 12, doppler processing circuitry 13, an image generation circuit 14, an image memory 15, a storage circuit 16, processing circuitry 17, and a network interface circuit 18.

The transmitter/receiver circuitry 11 is provided with a transmitter circuitry and a receiver circuitry. The transmitter/receiver circuitry 11 is controlled by the processing circuitry 17 and controls transmission directivity and reception directivity in transmission/reception of ultrasonic waves. While FIG.

1 shows an example in a case where the transmitter/receiver circuitry 11 is provided in the ultrasonic image processing apparatus 10, the transmitter/receiver circuitry 11 may be provided in the ultrasonic probe 20 or may be provided in both of the ultrasonic image processing apparatus 10 and the ultrasonic probe 20.

The transmitter circuitry includes a pulse generator, a transmission delay circuit, and a pulser circuit, and supplies drive signals to an ultrasonic transducer. The pulse generator repeatedly generates rate pulses for forming transmission ultrasonic waves at a prescribed rate frequency. The transmission delay circuit gives delay time for each piezoelectric transducer needed to converge the ultrasonic waves generated from the ultrasonic transducer into a beam shape and determine the transmission directivity, to each rate pulse generated by the pulse generator. The pulser circuit applies a drive pulse to the ultrasonic transducer at a timing based on the rate pulse. The transmission delay circuit adjusts any transmission direction of an ultrasonic beam transmitted from a piezoelectric transducer surface by changing the delay time to be given to each rate pulse.

The receiver circuitry includes an amplifier circuit, an A/D converter, and an adder, receives echo signals from the ultrasonic transducer, performs various kinds of processing to the echo signals, and generates echo data. The amplifier circuit amplifies the echo signals for each channel and performs gain correction processing. The A/D converter A/D converts the gain-corrected echo signals, and gives the delay time needed to determine the reception directivity to digital data. The adder performs addition processing of the echo signals processed by the A/D converter and generates the echo data. By the addition processing of the adder, a reflection component from a direction according to the reception directivity of the echo signals is emphasized.

The B mode processing circuitry 12 and the doppler processing circuitry 13 are signal processing circuitry. The B mode processing circuitry 12 receives the echo data from the receiver circuitry, executes logarithmic amplification, envelope detection processing, or the like, and generates B mode data which is data expressing signal strength of reflected waves by brightness of luminance. The doppler processing circuitry 13 frequency-analyzes speed information from the echo data received from the receiver circuitry, extracts a blood flow, tissue, and contrast medium echo components by a doppler effect, and generates doppler data which is data for which moving form information such as a flow rate, dispersion, and power, is extracted for multiple points.

The image generation circuit 14 generates ultrasonic images based on the data generated in the signal processing circuitry. For example, the image generation circuit 14 generates B mode images expressing the signal strength of the reflected waves by the luminance from two-dimensional B mode data generated by the B mode processing circuitry 12. For example, the image generation circuit 14 generates color doppler images indicating the moving form information from two-dimensional doppler data generated by the doppler processing circuitry 13. The image generation circuit 14 stores the generated ultrasonic images in the image memory 15. Further, the image generation circuit 14 stores the generated ultrasonic images in the storage circuit 16.

The image memory 15 is, for example, formed of a semiconductor memory such as a RAM, and stores the ultrasonic images generated by the image generation circuit 14. The image memory 15 stores the plurality of ultrasonic images along a time series tracing back to the past from the time of a freeze operation over one heartbeat or more of an object. In addition, the plurality of ultrasonic images stored in the image memory 15 are used for cine display. Here, the cine display is to display a plurality of still images along the time series at a fixed interval like moving images. Further, the image memory 15 can store the plurality of ultrasonic images collected in the past and read via the input interface 22 and the network interface circuit 18.

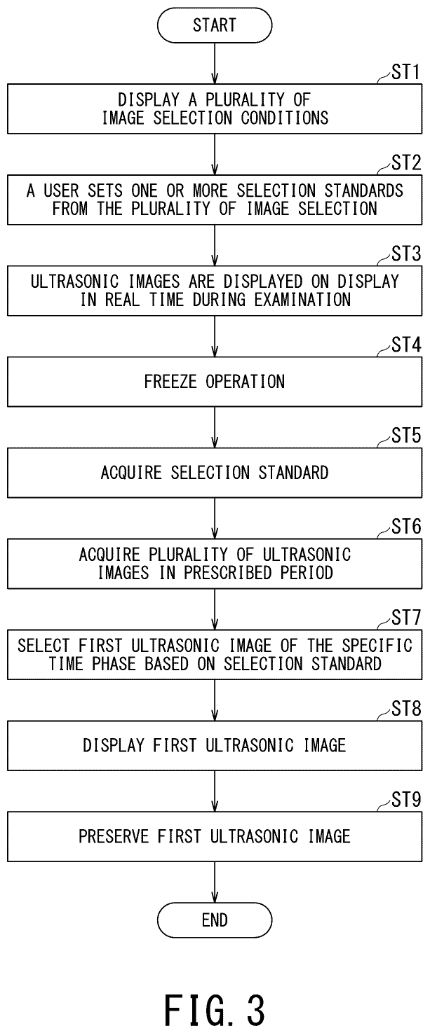

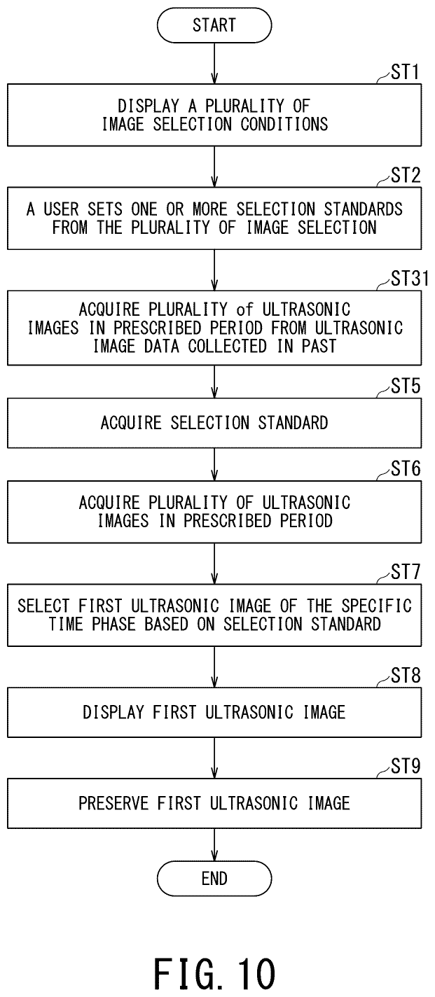

The storage circuit 16 includes, in addition to a transitory computer-readable medium, a non-transitory computer-readable medium having a computer program that realizes all or a part of functions F1 to F6 to be described later with FIG. 3 and FIG. 10 recorded hereon.

The storage circuit 16 is, for example, formed of a recording medium readable by a processor, such as a semiconductor memory element like a RAM (Random Access Memory) or a flash memory, a hard disk, or an optical disk. The storage circuit 16 stores various kinds processing programs, which may include an OS (Operating System) in addition to an application program, used in the processing circuitry 17, and data required for execution of the program. A part or all of the programs and the data in a storage medium in the storage circuit 16 may be downloaded by communication via a network, or may be supplied to the storage circuit 16 via a portable storage medium such as an optical disk. Note that a part or all of information stored in the storage circuit 16 may be distributed and stored in at least one of the storage media such as an external storage circuit and an unshown storage circuit of the ultrasonic probe 20, or may be duplicated and stored.

The processing circuitry 17 includes an exclusive or general purpose processor, and realizes a function of generally controlling the ultrasonic diagnostic device 1. The processing circuitry 17 reads and deploys a control program stored in the storage circuit 16 on a memory, and controls various kinds of units of the ultrasonic diagnostic device 1 according to the deployed control program. In addition, the processing circuitry 17 realizes various kinds of functions to be described later by reading and executing an ultrasonic image processing program stored in the storage circuit 16, for example.

The network interface circuit 18 is mounted with various information communication protocols according to a form of the network. The network interface circuit 18 connects the ultrasonic image processing apparatus 10 and other electric apparatuses according to the various kinds of protocols. For the connection, electric connection via an electronic network can be applied. Here, the term electronic network refers to an information communication network in general utilizing telecommunication technology, and includes a wireless/cable LAN (Local Area Network) such as a hospital basic LAN, an internet network, a telephone communication network, an optical fiber communication network, a cable communication network, and a satellite communication network or the like.

The ultrasonic probe 20 includes a plurality of ultrasonic transducers (piezoelectric transducers) disposed in an array shape. The ultrasonic probe 20 is freely detachably connected with the ultrasonic image processing apparatus 10 via a cable. The ultrasonic probe 20 may be wirelessly connected with the ultrasonic image processing apparatus 10.

The display 21 is formed of a general display device such as a liquid crystal display and an OLED (Organic Light Emitting Diode) display. The display 21 is a display device and may also include a GUI (Graphic User Interface) or a touch panel capable of accepting operations, for example. The display 21 is controlled by the processing circuitry 17 and displays various kinds of images and various kinds of information. The display 21 is an example of a display unit.

The input interface 22 includes an input device that is operable, and an input circuit that inputs signals from the input device. The input device is realized by a mouse, a keyboard, a track ball, a switch, a button, a joystick, a touch pad with which an input operation is performed by touching an operation surface, a touch screen for which a display and a touch pad are integrated, a non-contact input circuit using an optical sensor, or a voice input circuit or the like. When the input device accepts an input operation, the input circuit generates an electric signal according to the input operation and outputs it to the processing circuitry 17.

The input interface 22 is mounted with a portable memory such as a USB memory, a memory card, a magnetic disk, or an optical disk, and includes a circuit that inputs the various kinds of data stored in the portable memories. The input interface 22 is an example of an input unit.

The electrocardiograma connected to the ultrasonic image processing apparatus 10 via the network interface circuit 18. The electrocardiograma measuring device that detects electric signals (that is, ECG: electrocardiogram signals) generated in synchronization with the heartbeat from an electrode attached to the object. Information (for example, ECG waveforms) of an electrocardiogram by the ECG signals detected by the electrocardiograph on the display 21. The information of the electrocardiogram is used for determining a timing of the freeze operation and selecting the ultrasonic image of a specific time phase to be described later, for example. The information of the electrocardiogram may be information of similar biological waveforms obtained by a unit other than the electrocardiogra

### First Embodiment

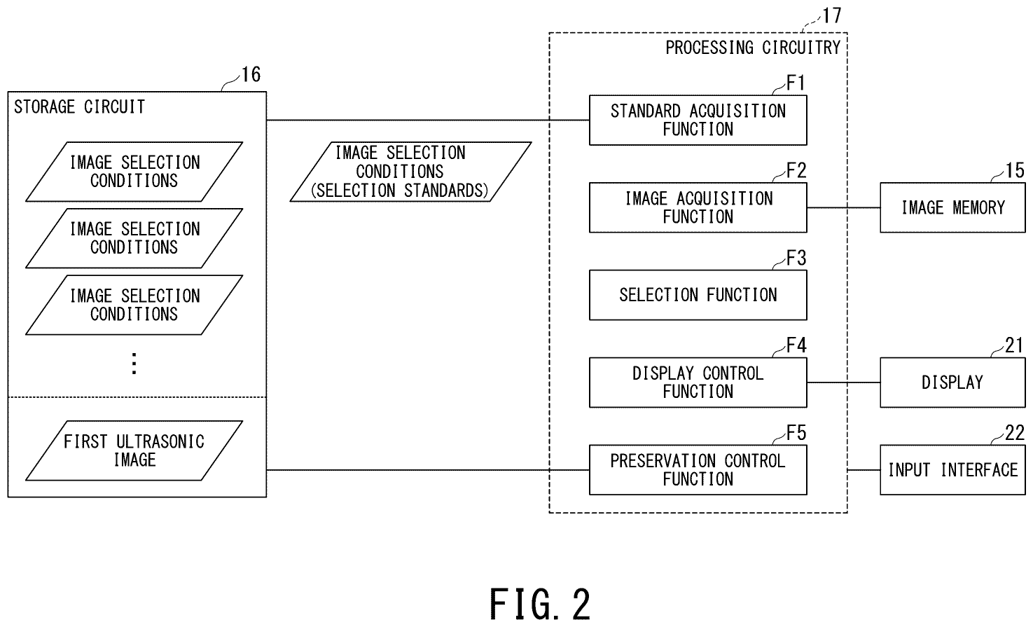

FIG. 2 is a block diagram showing a configuration example of the processing circuitry 17 of the ultrasonic image processing apparatus 10 according to the first embodiment. As shown in FIG. 2, the processing circuitry 17 in the first embodiment realizes a standard acquisition function F1, an image acquisition function F2, a selection function F3, a display control function F4, and a preservation control function F5. FIG. 3 is a flowchart showing an operation example of the ultrasonic image processing apparatus 10 according to the first embodiment.

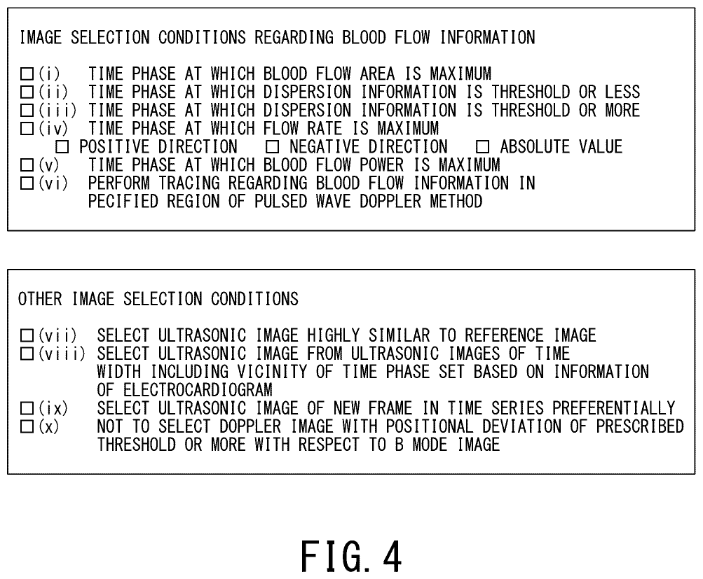

In step ST1, the display control function F4 performs control to display a plurality of image selection conditions on the display 21 so that at least one of the plurality of image selection conditions as candidates of a selection standard can be selected. FIG. 4 is an explanatory diagram showing the plurality of image selection conditions displayed on the display 21. Note that the control may be performed so as to display a part or all of the plurality of image selection conditions exemplified in FIG. 4 on the display 21 according to an imaging region and an examination content.

The image selection conditions are the candidates of the selection standard which is a standard for selecting a first ultrasonic image (still image) of the specific time phase. The image selection conditions may be, for example, as shown in an upper stage in FIG. 4, conditions regarding blood flow information, such as (i) a time phase at which blood flow area is maximum, (ii) a time phase at which dispersion information is a threshold or below, (iii) a time phase at which the dispersion information is the threshold or above, (iv) a time phase at which a flow rate is maximum in any one of a positive direction, a negative direction, or an absolute value, and (v) a time phase at which blood flow power is maximum. Further, the image selection conditions may include a condition that determines (vi) to perform tracing regarding the blood flow information in a specified region of a pulsed wave doppler method (PWD).

Additionally, the image selection condition may be, as shown in a lower stage in FIG. 4, a condition that determines (vii) to select the ultrasonic image highly similar to a reference image as the first ultrasonic image. The image selection condition may be a condition that determines (viii) to select the first ultrasonic image from the ultrasonic images of a time width including a vicinity of the time phase set based on the information of the electrocardiogram. The image selection condition may be a condition that determines (ix) to preferentially select the ultrasonic image of a new frame in the time series as the first ultrasonic image. The image selection condition may be a condition that determines (x) not to select a doppler image with a positional deviation of a prescribed threshold or above with respect to the B mode image.

In step ST2, a user such as an examiner or an engineer sets one or more selection standards from the plurality of image selection conditions that are the candidates of the selection standard, that are displayed on the display 21, via the input interface 22. Note that the selection standard may be set for each workflow or each examination.

In step ST3, the ultrasonic images are displayed on the display 21 in real time during the examination. The ultrasonic images are collected in a doppler mode of observing the blood flow information, for example.

In step ST4, the freeze operation is performed during the examination. When the ultrasonic image of the specific time phase suitable for the imaging region and the examination content is displayed on the display 21, the user performs the freeze operation by pressing a button for switching freeze and release. The freeze operation is an operation for stopping display of the ultrasonic images acquired in real time during the examination. The plurality of ultrasonic images along the time series tracing back to the past from the time of the freeze operation are stored in the image memory 15.

The ultrasonic image processing apparatus 10 starts ultrasonic image processing according to the embodiment at the timing of the freeze operation during the examination. That is, the processing circuitry 17 acquires the plurality of ultrasonic images in the prescribed period when performing a freeze operation of stopping display of ultrasonic images acquired in real time. The standard acquisition function F1, the image acquisition function F2, and the selection function F3 may start a standard acquiring operation, an image acquiring operation, and a selecting operation at the timing of the freeze operation. Note that the user may preset whether or not to perform the ultrasonic image processing via the input interface 22. In the case of not performing the ultrasonic image processing, for example, the ultrasonic image displayed on the display 21 at the time of the freeze operation is displayed as the still image.

In step ST5, the standard acquisition function F1 acquires the selection standard to be the standard of selecting the first ultrasonic image of the specific time phase. The first ultrasonic image is the still image of one frame or a plurality of frames.

The standard acquisition function F1 acquires one or more selection standards set from the plurality of image selection conditions that are the candidates of the selection standard, for example. The standard acquisition function F1 acquires the selection standard regarding the blood flow information that is at least one of (i) the time phase at which the blood flow area is maximum, (ii) the time phase at which the dispersion information is the threshold or below, (iii) the time phase at which the dispersion information is the threshold or above, (iv) the time phase at which the flow rate is maximum in any one of the positive direction, the negative direction, or the absolute value, and (v) the time phase at which the blood flow power is maximum, as shown in the upper stage in FIG. 4, for example.

The standard acquisition function F1 may acquire the image selection condition that determines to perform the tracing regarding the blood flow information in the specified region of the pulsed wave doppler method as the selection standard. The standard acquisition function F1 may acquire the image selection condition that determines to select an image highly similar to the reference image as the selection standard. The standard acquisition function F1 may select the image selection condition as the selection standard that determines to select the image from the ultrasonic images of the time width including the vicinity of the time phase set based on the information of the electrocardiogram. The standard acquisition function F1 may acquire the image selection condition as the selection standard that determines to preferentially select the ultrasonic image of a new frame in the time series. The standard acquisition function F1 may acquire the image selection condition as the selection standard that determines not to select the doppler image with the positional deviation of the prescribed threshold or above with respect to the B mode image.

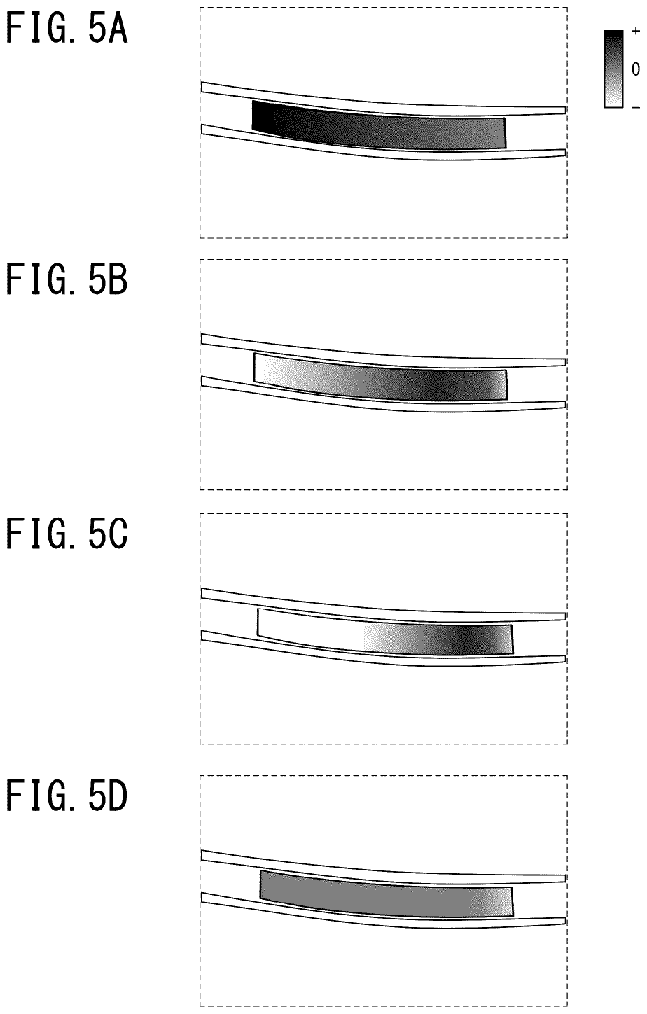

In step ST6, the image acquisition function F2 acquires the plurality of ultrasonic images in a prescribed period from the plurality of ultrasonic images along the time series. The plurality of ultrasonic images in the prescribed period are, for example, the plurality of doppler images generated in the doppler mode of observing the blood flow information, as shown in FIG. 5A to FIG. 5D to be described later. The plurality of ultrasonic images in the prescribed period are acquired from the image memory 15, for example.

The prescribed period is a period over at least one heartbeat of the object. The prescribed period is set based on at least one of the information of the electrocardiogram, the blood flow information, and information specified by the user.

For example, a period of the time width in the vicinity of the specific time phase may be set as the prescribed period based on the information of the electrocardiogram. In addition, a period for the plurality of heartbeats may be set as the prescribed period according to the imaging region and the examination content. For example, the period for three heartbeats may be the prescribed period in the case of an artery examination, and the period for two heartbeats may be the prescribed period in the case of a circulatory organ inspection.

In the case of setting the prescribed period based on the blood flow information, the period for one heartbeat or more during which the tracing of the blood flow information such as the flow rate, the dispersion, and the power is stable may be set as the prescribed period. For example, in the case where the tracing of the blood flow information is unstable due to irregular pulses or the like, the period for a plurality of heartbeats may be set as the prescribed period. In the case where the period having stable tracing of the blood flow information is shorter than one heartbeat, the ultrasonic image processing according to the embodiment may not be performed.

Further, the user may specify the prescribed period via the input interface 22 such as a touch panel screen and operation panel.

In step ST7, the selection function F3 selects the first ultrasonic image from the plurality of ultrasonic images in the prescribed period based on the selection standard. That is, the selection function F3 selects the first ultrasonic image of the specific time phase from the plurality of ultrasonic images in the prescribed period based on the selection standard which is preset For example, the first ultrasonic image is the doppler image of FIG. 5A selected from FIG. 5A to FIG. 5D that are the plurality of doppler images in the prescribed period to be described later.

For example, when the image selection condition (vi) shown in the upper stage in FIG. 4 is selected, the selection function F3 performs the tracing of the blood flow information based on the selection standard in each of the plurality of ultrasonic images in the prescribed period, extracts the specific time phase based on a result of the tracing, and selects the first ultrasonic image corresponding to the extracted specific time phase.

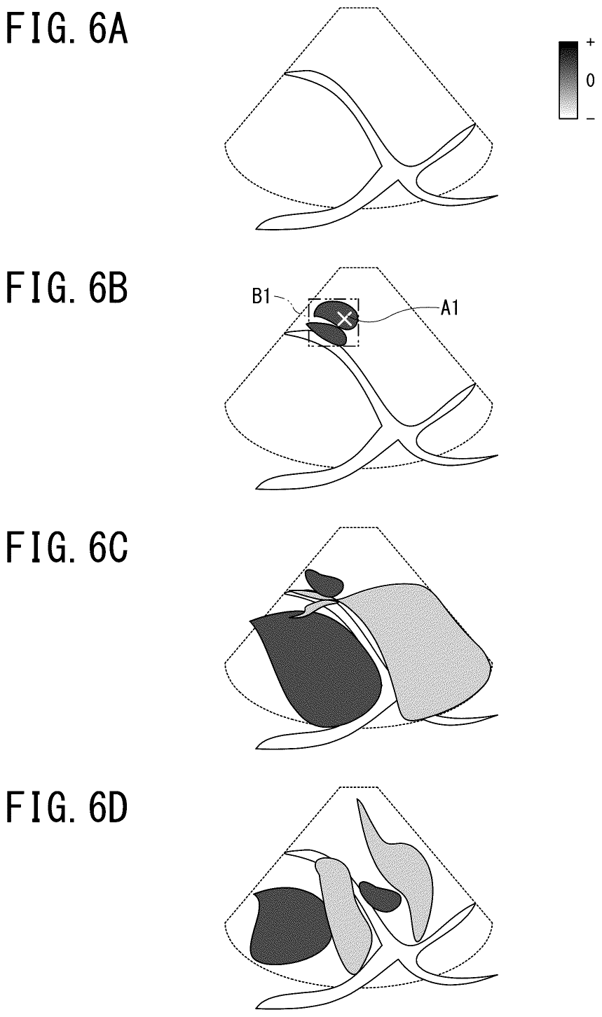

In this case, the selection function F3 may perform the tracing of the blood flow information based on the selection standard in the specified region specified in the plurality of ultrasonic images in the prescribed period. For example, the specified region is specified by the user pointing out a cursor position (for example, A1 in FIG. 6B) or setting a specific region (for example, B1 in FIG. 6B) in the ultrasonic image displayed on the display 21. By specifying a region to perform the tracing of the blood flow information, the first ultrasonic image of the specific time phase can be more highly accurately selected.

Further, when the image selection condition (viii) shown in the lower stage in FIG. 4 is selected, the selection function F3 may select the first ultrasonic image from the plurality of ultrasonic images in the prescribed period which is the time width including the vicinity of the specific time phase set based on the information of the electrocardiogram. Since the plurality of ultrasonic images in the prescribed period are limited to the time width including the vicinity of the specific time phase, the first ultrasonic image of the specific time phase can be more highly accurately selected.

When the image selection condition (vii) shown in the lower stage in FIG. 4 is selected, the selection function F3 may select the ultrasonic image highly similar to the reference image among the plurality of ultrasonic images in the prescribed period as the first ultrasonic image. The reference image may be the ultrasonic image of the specific time phase in the present examination, or may be the ultrasonic image of the specific time phase collected in the past examination.

When the image selection condition (ix) shown in the lower stage in FIG. 4 is selected, the selection function F3 may preferentially select the ultrasonic image of a new frame in the time series among the plurality of ultrasonic images in the prescribed period as the first ultrasonic image. In this case, the first ultrasonic image may be selected while tracing back from the latest (for example, the time of the freeze operation) frame to old frames in the time series. Further, a priority index may be added to each of the plurality of selection standards, and the ultrasonic image satisfying the selection standard having the highest priority index may be preferentially selected as the first ultrasonic image. In this case, the first ultrasonic image is selected in an order from the frame satisfying the selection standard having the highest priority index to the frame satisfying the selection standard having the low priority index.

When the image selection condition (x) shown in the lower stage in FIG. 4 is selected, the selection function F3 does not need to select the doppler image having the positional deviation of the prescribed threshold or above with respect to the B mode image as the first ultrasonic image. By extracting a correlation with the latest B mode image in the time series and feature points, the doppler images having the imaging region different from that of the B mode image are not necessarily to be selected as the first ultrasonic image even if they are the plurality of ultrasonic images in the prescribed period.

In step ST8, the display control function F4 performs control so as to display the first ultrasonic image on the display 21. In the case where the plurality of first ultrasonic images are present, thumbnail display may be performed so that all the first ultrasonic images on the display 21 can be visually recognized. The plurality of first ultrasonic images may be displayed in the case of there are the plurality of first ultrasound images of each the specific time phases corresponding to each the selection standards by being acquired a plurality of the selection standards in step ST5 and, for example.

In step ST9, the preservation control function F5 performs control so as to preserve the first ultrasonic image in the storage circuit 16.

Here, a selection example of the first ultrasonic image at a specific imaging region will be described using FIG. 5A to FIG. 6D. Note that color doppler images are generally represented by red indicating the blood flow toward a probe, blue indicating the blood flow moving away from the probe and hue in-between, but are represented by a gray scale with the red as +side black and with the blue as-side white in FIG. 5A to FIG. 6D.

FIG. 5A to FIG. 5D are explanatory diagrams about selection of the first ultrasonic image in a carotid artery. In the ultrasonic image showing the carotid artery, for example, even in a state where breathing is temporarily stopped, the blood flow information changes in order along FIG. 5A to FIG. 5D due to pulsation. In this case, there may be a time lag between a timing at which the user wants to perform preservation as the still image and the timing that the user performs the freeze operation. Because of the freeze operation, the ultrasonic images (for example, FIG. 5B to FIG. 5D) shifted from the ultrasonic image (for example, FIG. 5A) of the specific time phase may be displayed. Then, when the time phase at which the blood flow area is maximum and the time phase at which the dispersion information is the threshold or below are defined as the selection standards, the ultrasonic image of the specific time phase in the past having the maximum blood flow area without folding is selected as the first ultrasonic image (FIG. 5A). In this case, the time phase at which the blood flow area is maximum in the specific region or a lumen structure may be defined as the selection standard.

FIG. 6A to FIG. 6D are explanatory diagrams about the selection of the first ultrasonic image in a circulatory organ. In the ultrasonic image in the circulatory organ, for example, the blood flow information changes in order along FIG. 6A to FIG. 6D. In this case, in the ultrasonic image in the circulatory organ, a reverse flow is sometimes evaluated as a standard to determine whether or not a valve function is normal. Then, by defining the time phase at which the dispersion information is the threshold or above in the specified region of the pulsed wave doppler method or in the entire region as the selection standard, the ultrasonic image (for example, FIG. 6B) of the specific time phase in the past that is not a normal blood flow time phase but a reverse flow time phase is selected as the first ultrasonic image. In this case, the first ultrasonic image may be selected from the plurality of ultrasonic images of the time width including the vicinity of the time phase set based on the information of the electrocardiogram.

According to the ultrasonic image processing apparatus 10 according to the first embodiment, when the user performs the freeze operation in real time during scanning, the first ultrasonic image of the specific time phase is selected based on the selection standard. Since the specific time phase is automatically traced back, the first ultrasonic image is displayed as the still image, and the first ultrasonic image of the specific time phase can be easily preserved, examination efficiency is improved. In addition, since dispersion of the selected still image caused by the user's manual selection depending on the user's subjectivity and experience level is reduced, examination accuracy is improved.

### (Modification of First Embodiment)

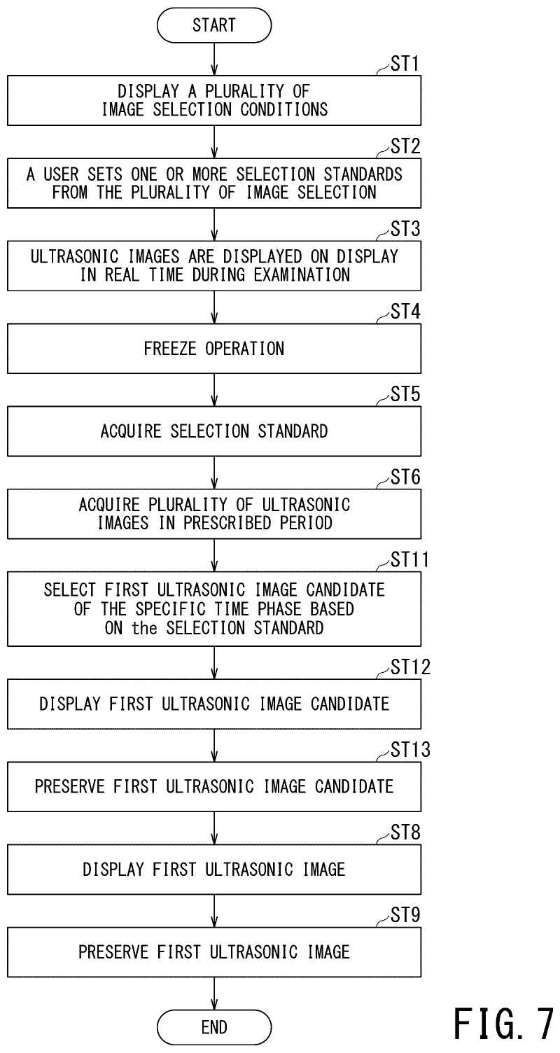

FIG. 7 is a flowchart showing an operation example of the ultrasonic image processing apparatus 10 according to the modification of the first embodiment. The modification of the first embodiment differs from the first embodiment in that the ultrasonic images in the time series before and after the ultrasonic image selected as a first ultrasonic image candidate based on the selection standard can be selected as the first ultrasonic images. In the modification of the first embodiment, processing advances to step ST11 after step

ST6.

In step ST11, the selection function F3 selects the first ultrasonic image candidate from the plurality of ultrasonic images in the prescribed period based on the selection standard. Step ST11 is not practically different from step ST7 except that, while the selection function F3 selects the “first ultrasonic image” corresponding to the specific time phase from the plurality of ultrasonic images in the prescribed period based on the selection standard in step ST7, the “first ultrasonic image candidate” corresponding to the specific time phase is selected in step ST11, so that redundant description is omitted. In step ST11 in the modification of the first embodiment, the provisional “first ultrasonic image candidate” is selected by the processing similar to that in step ST7, and the final “first ultrasonic image” is selected in subsequent step ST13.

In step ST12, the display control function F4 performs control so as to display the first ultrasonic image candidate on the display 21. Along with that, the display control function F4 performs control so as to switch the display of the first ultrasonic image candidate to the display of the ultrasonic images in the time series before and after the first ultrasonic image candidate, according to an input operation by the user. Thus, the user can perform fine adjustment from the first ultrasonic image candidate to the ultrasonic images in the time series before and after the candidate. For example, via the input interface 22 such as a track ball, switching is performed to the display of the desired ultrasonic images that are the ultrasonic images in the time series before and after the first ultrasonic image candidate.

In step ST13, the selection function F3 defines the ultrasonic image after the switch of the display as the first ultrasonic image.

According to the ultrasonic image processing apparatus 10 according to the modification of the first embodiment, even in the case where the first ultrasonic image candidate is different from a desired ultrasonic image that the user wants to display or preserve as the still image, the first ultrasonic image can be selected. In this case, since the first ultrasonic image is finally selected from the first ultrasonic image candidates which are frames close to the first ultrasonic image, operation time can be suppressed and the examination efficiency is improved.

### Second Embodiment

A configuration example of the ultrasonic diagnostic device 1 including the ultrasonic image processing apparatus 10 according to the second embodiment is same as the configuration example of the ultrasonic diagnostic device 1 including the ultrasonic image processing apparatus 10 according to the first embodiment shown in FIG. 1, so that the description is omitted.

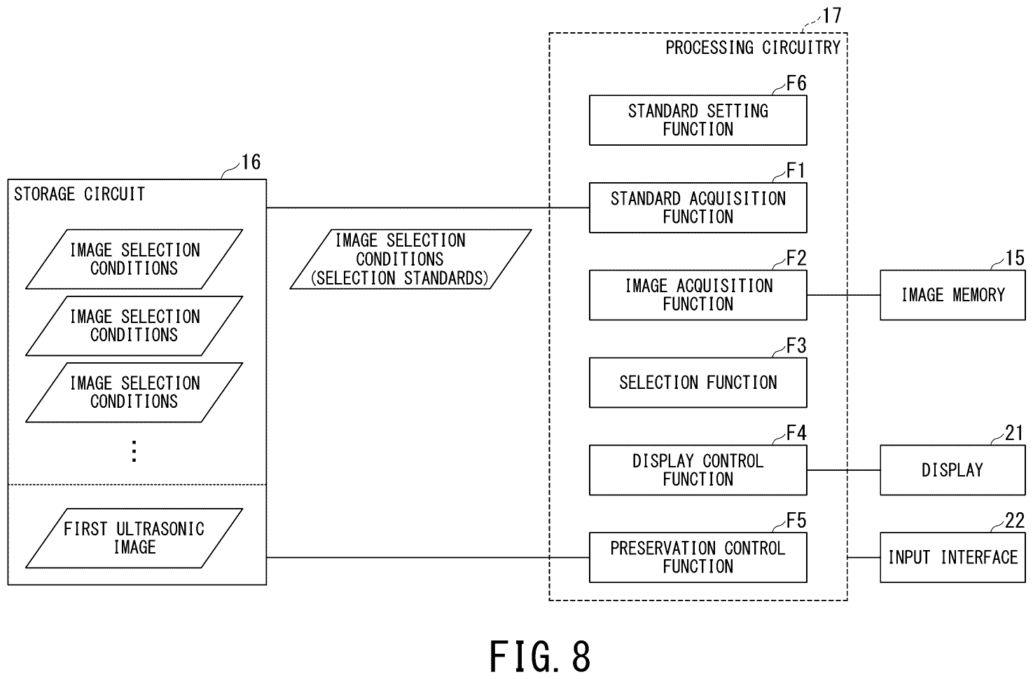

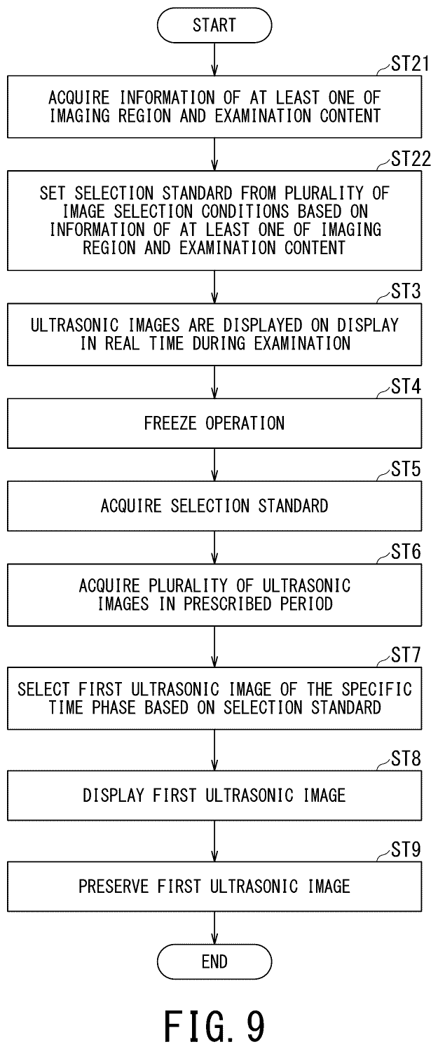

FIG. 8 is a block diagram showing a configuration example of the processing circuitry 17 of the ultrasonic image processing apparatus 10 according to the second embodiment. As shown in FIG. 8, the processing circuitry 17 in the second embodiment further realizes a standard setting function F6. FIG. 9 is a flowchart showing an operation example of the ultrasonic image processing apparatus 10 according to the second embodiment. The second embodiment in which the selection standard is set from the image selection conditions based on the information of at least one of the imaging region and the examination content differs from the first embodiment in which the user sets the selection standard from the image selection conditions. In the second embodiment, the processing is started from step ST21.

In step ST21, the standard setting function F6 acquires the information of at least one of the imaging region and the examination content. The information of the imaging region and the examination content is included in an imaging condition protocol read from the storage circuit 16 and an imaging condition input in the input interface 22.

In step ST22, the standard setting function F6 sets the selection standard from the plurality of image selection conditions based on the information of at least one of the imaging region and the examination content. For example, in the case where the imaging region is the circulatory organ and the examination content is reverse flow evaluation, the selection standard is set to select the time phase at which the dispersion information is the threshold or above in the specified region of the pulsed wave doppler method as the first ultrasonic image from the plurality of ultrasonic images of the time width including the vicinity of the time phase set based on the information of the electrocardiogram. Without being limited to this selection standard setting example, the selection standard can be set according to the information of at least one of the clinically known imaging region and examination content. Association data in which the information of at least one of the imaging region and the examination content and the selection standard are associated may be stored in the storage circuit 16 for example, and the selection standard may be set using the association data.

In the second embodiment, after step ST22, the processing advances to step ST3. In step ST5 in the first embodiment, the standard acquisition function F1 acquires the selection standard set by the user. In contrast, in step ST5 in the second embodiment, the standard acquisition function F1 acquires the selection standard set based on the information of at least one of the imaging region and the examination content. According to the ultrasonic image processing apparatus 10 according to the second embodiment, the first ultrasonic image of the specific time phase is selected even in the case where the user does not set the selection standard.

### Third Embodiment

A configuration example of the ultrasonic diagnostic device 1 including the ultrasonic image processing apparatus 10 according to the third embodiment is the same as the configuration example of the ultrasonic diagnostic device 1 including the ultrasonic image processing apparatus 10 according to the first embodiment shown in FIG. 1, so that the description is omitted. In addition, the configuration example of the processing circuitry 17 of the ultrasonic image processing apparatus 10 according to the third embodiment is the same as the configuration example of the processing circuitry 17 of the ultrasonic image processing apparatus 10 according to the first embodiment shown in FIG. 2, so that the description is omitted.

FIG. 10 is a flowchart showing an operation example of the ultrasonic image processing apparatus 10 according to the third embodiment. In the third embodiment, the plurality of ultrasonic images in the prescribed period are acquired by a data read operation on the plurality of ultrasonic images collected in the past, while in the first embodiment, the plurality of ultrasonic images in the prescribed period are acquired by the freeze operation in real time during an examination, which are different. In the third embodiment, after step ST2, the processing advances to step ST31.

In step ST31, the plurality of ultrasonic images in the prescribed period are acquired from ultrasonic image data collected in the past. The ultrasonic image data collected in the past is acquired via the storage circuit 16, the input interface 22, and the network interface circuit 18, for example. The ultrasonic image data collected in the past is read to the image memory 15. The processing circuitry 17 acquires the plurality of ultrasonic images in the prescribed period when a data read operation of reading a plurality of ultrasonic images collected in past is completed. The standard acquisition function F1, the image acquisition function F2, and the selection function F3 may start the standard acquiring operation, the image acquiring operation, and the selecting operation at a timing at which the data read operation of the plurality of ultrasonic images collected in the past is completed.

In the third embodiment, after step ST31, the processing advances to step ST5. According to the ultrasonic image processing apparatus 10 according to the third embodiment, the first ultrasonic image of the specific time phase can be selected using the ultrasonic image data collected in the past.

According to the ultrasonic image processing apparatus, the non-transitory computer-readable storage medium storing the ultrasonic image processing program, and the ultrasonic image processing method in at least one embodiment described above, the ultrasonic image of the specific time phase suitable for the imaging region and the examination content can be selected as the still image. Thus, the still image of the ultrasonic image of the specific time phase suitable for the imaging region and the examination content can be preserved as an image for diagnostic use.

In the above-described embodiments, the term “processor” means a circuit such as a special-purpose or general-purpose CPU (Central Processing Unit), a GPU (Graphics Processing Unit), an ASIC (Application Specific Integrated Circuit), a programmable logic device including an SPLD (Simple Programmable Logic Device) and a CPLD (Complex Programmable Logic Device), and an FPGA (Field Programmable Gate Array), for example. When the processor is a CPU, for example, the processor implements various functions by reading in and executing the programs (the medical image processing program) stored in the memory. In addition, when the processor is an ASIC, for example, instead of storing the programs in the memory, the functions corresponding to the programs are directly incorporated as a logic circuit in the circuit of the processor. In this case, the processor implements the various functions by hardware processing in which the processor reads out and executes the programs incorporated in the circuit. Additionally or alternatively, the processor can implement the various functions by combining the software processing and the hardware processing.

Although a description has been given of the case where a single processor of the processing circuitry achieves the respective functions in the above-described embodiments, the processing circuitry may be configured by combining a plurality of independent processors so that each processor implements each function. Further, when a plurality of processors is provided, a memory for storing the programs may be provided for each processor or a single memory may collectively store the programs corresponding to the functions of all the processors.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the scope of the inventions as defined by the appended claims.

What is claimed is:

1. An ultrasonic image processing apparatus comprising processing circuitry configured to:
acquire a plurality of ultrasonic images in a prescribed, and
select a first ultrasonic image of a specific time phase from the plurality of ultrasonic images in the prescribed period based on a selection standard which is preset.

2. The ultrasonic image processing apparatus according to claim 1,
wherein the processing circuitry is configured to acquire the plurality of ultrasonic images in the prescribed period from a plurality of ultrasonic images along a time series.

3. The ultrasonic image processing apparatus according to claim 1,
wherein the processing circuitry is configured to acquire the plurality of ultrasonic images in the prescribed period when performing a freeze operation of stopping display of ultrasonic images acquired in real time or when a data read operation of reading a plurality of ultrasonic images collected in past is completed.

4. The ultrasonic image processing apparatus according to claim 1,
wherein the plurality of ultrasonic images in the prescribed period is doppler images generated in a doppler mode for observing blood flow information.

5. The ultrasonic image processing apparatus according to claim 1,
wherein the processing circuitry is configured to perform tracing of blood flow information based on the selection standard in each of the plurality of ultrasonic images in the prescribed period, extract the specific time phase based on a result of the tracing, and select the first ultrasonic image corresponding to the extracted specific time phase.

6. The ultrasonic image processing apparatus according to claim 1,
wherein the processing circuitry is configured to acquire the selection standard regarding blood flow information that is at least one of (a) a time phase at which blood flow area is maximum, (b) a time phase at which dispersion information is a threshold or below, (c) a time phase at which the dispersion information is the threshold or above, (d) a time phase at which a flow rate is maximum in any one of a positive direction, a negative direction, or an absolute value, and (e) a time phase at which blood flow power is maximum.

7. The ultrasonic image processing apparatus according to claim 5,
wherein the processing circuitry is configured to perform the tracing in a specific region specified in the plurality of ultrasonic images in the prescribed period.

8. The ultrasonic image processing apparatus according to claim 1,
wherein the processing circuitry is configured to define a time width including a vicinity of a time phase set based on information of an electrocardiogram as the prescribed period, and select the first ultrasonic image from the plurality of ultrasonic images in the prescribed period.

9. The ultrasonic image processing apparatus according to claim 1,
wherein the processing circuitry is configured to select an ultrasonic image highly similar to a reference image among the plurality of ultrasonic images in the prescribed period as the first ultrasonic image.

10. The ultrasonic image processing apparatus according to claim 1,
wherein the prescribed period is a period set based on at least one of information of an electrocardiogram, blood flow information, and information specified by a user.

11. The ultrasonic image processing apparatus according to claim 2,
wherein the processing circuitry is configured to preferentially select an ultrasonic image of a new frame in the time series among the plurality of ultrasonic images in the prescribed period as the first ultrasonic image.

12. The ultrasonic image processing apparatus according to claim 4,
wherein the processing circuitry is configured not to select a doppler image having a positional deviation of a prescribed threshold or above with respect to a B mode image as the first ultrasonic image.

13. The ultrasonic image processing apparatus according to claim 1,
wherein the processing circuitry is further configured to perform control so as to display the first ultrasonic image on a display.

14. The ultrasonic image processing apparatus according to claim 1,
wherein the processing circuitry is further configured to perform control so as to preserve the first ultrasonic image in a storage circuit.

15. The ultrasonic image processing apparatus according to claim 1,
wherein the processing circuitry is further configured to perform control so as to display a plurality of image selection conditions as candidates of the selection standard on a display such that at least one of the plurality of image selection conditions can be selected, and acquire the selection standard set according to an input operation by a user from the plurality of image selection conditions.

16. The ultrasonic image processing apparatus according to claim 1,
wherein the processing circuitry is further configured to perform control so as to display a candidate of the first ultrasonic image on a display, and perform control so as to switch display of the candidate of the first ultrasonic image to display of the ultrasonic images in the time series before and after the candidate of the first ultrasonic image according to an input operation by a user, and
define the ultrasonic image after switch of the display as the first ultrasonic image.

17. The ultrasonic image processing apparatus according to claim 1,
wherein the processing circuitry is further configured to set the selection standard from a plurality of image selection conditions based on information of at least one of an imaging region and an examination content.

18. A non-transitory computer-readable storage medium storing an ultrasonic image processing program, the program being for executing:
a step of acquiring a plurality of ultrasonic images in a prescribed period; and
a step of selecting a first ultrasonic image of a specific time phase from the plurality of ultrasonic images in the prescribed period based on a selection standard which is set.

19. An ultrasonic image processing method comprising:
acquiring a plurality of ultrasonic images in a prescribed period; and
selecting a first ultrasonic image of a specific time phase from the plurality of ultrasonic images in the prescribed period based on a selection standard which is preset.
